(19) United States
(12) Patent Application Publication
(10) Pub. No.: US 2008/0195085 A1
(43) Pub. Date: Aug. 14, 2008

(54) Economical, two component, thermal energy delivery and surface cooling apparatus and its method of use

(76) Inventor: Marvin P. Loeb, Huntington Beach, CA (US)

Correspondence Address: IP FOCUS LAW GROUP, LTD, 608 NORTH CARLYLE LANE, SUITE 100, ARLINGTON HEIGHTS, IL, 60004, US

(21) Appl. No.: 11/370,659

(22) Filed: Mar. 7, 2006

(51) Int. Cl. A61B 18/20

(52) U.S. Cl. 606/3; 606/33

(57) ABSTRACT

The present invention is embodied in a medical device which is comprised of a thermal energy delivery component, for example, including an elongate optical fiber terminating in a lateral laser energy emitter, and an outer coolant component, which includes a cannula for receiving the thermal energy delivery component, which terminates in an energy-transmissive balloon for surrounding the thermal energy emitter and providing a tissue-contacting coolant chamber. The cannula portion of the coolant component is moveably sealed around the laser energy delivery component. In one embodiment, a retaining means prevents the thermal energy delivery component from being detached from the coolant component. In an alternate embodiment, there is no retaining means, allowing the more costly thermal energy delivery component to be removed, sterilized and later reused, whereas the less costly outer coolant component, which contacts tissue, blood and body liquids, can be discarded after use.

### FIELD OF INVENTION

This invention relates to thermal energy delivery devices which are used to denature, shrink, coagulate, scar, desiccate or vaporize internal body tissues surrounding or underlying a duct, blood vessel, hollow-organ or body cavity, while concomitantly cooling the interior surface of the duct, blood vessel, hollow organ or body cavity to prevent damage to its endothelial lining, in an economical, minimally invasive procedure.

### BACKGROUND OF THE INVENTION

Thermal energy delivery devices include those emitting coherent light or laser energy, incoherent high intensity white light, incoherent high intensity light of a particular wavelength, microwave and focused ultra-sound energy. Of these, optical fibers for conveying laser energy, sometimes referred to as wave guides, enjoy certain advantages.

Fiber-optic based devices for delivering laser energy have many uses in medicine, as optical fibers are small in diameter, can reach areas of the body difficult to access by other means and can be made to emit laser energy straight ahead, sideways or at a desired angle. However, fiber-optic based devices are relatively expensive, particularly those which are able to emit laser energy laterally from the axis of the optical fiber at an angle of about 70° to 90°, generally referred to as side-firing laser devices.

For example, side-firing laser devices manufactured by Trimedyne, Inc., the owner of this application, which are used in minimally invasive, outpatient procedures, sell for $600 or more each. Such devices are sold as “single-use,” disposable devices, as they contact body tissue and blood and cannot be safely sterilized and re-used.

In the United States, where Medicare, insurance companies and health plans pay about $2,000 to $4,000 for a minimally invasive, outpatient medical procedure, the price of such devices and the cost of amortizing the equipment used in such procedures can be afforded. The same is true in Japan, where payments for medical procedures are also relatively high. However, in the developed countries of Europe, where only about $1,000 to $2,000 is paid for such procedures, such devices cannot presently be afforded, as the cost of such devices, operating room and nursing time and other supplies, as well as amortization of the equipment used in such procedures, cost more in aggregate than the amount paid. In underdeveloped countries, where the patients can pay only about $500 to $1,500 for a medical procedure, open surgery, using stainless steel scalpels and other utensils, is presently the only available choice for many patients.

There are many conditions which could be treated if laser energy could be applied to internal tissues underlying a duct, blood vessel, hollow organ or body cavity without damaging their endothelial lining, much as laser energy is used with concomitant cooling of the skin to shrink the underlying tissues, for example, to remove facial wrinkles, coagulate blood vessels or damage hair follicles. Common methods to cool the skin during the emission of laser energy in cosmetic procedures include the concomitant emission of a cryogenic gas, a water spray, cooled air, room temperature air or a cold gel, which is transparent to the wavelength of laser energy used. However, there presently exists no means to economically provide cooling to the endothelial lining of internal ducts, blood vessels, hollow-organs or body cavities, while laser energy passes through and creates the desired effect on underlying tissues.

Some of the conditions that may be so treated are gastro-esophageal reflux disease or GERD, female stress urinary incontinence or FSUI, fecal (anal) incontinence, mitral valve prolapse, benign prostatic hyperplasia or BPH, commonly referred to as an enlarged prostate, or an abdominal aortic aneurysm, where denaturing, shrinking, scarring, coagulating, desiccating or vaporizing the tissue underlying the endothelial lining could treat the condition, but where damage to the sensitive endothelial lining could cause pain and the risk of infection.

It would be desirable to be able to provide the benefit of thermal energy delivering devices to treat such conditions, without damaging the sensitive endothelial linings of internal body structures, as a device with two, non-detachable, components, one for delivery of thermal energy and one for cooling the lining, which is intended to be used once and discarded. Such a device could be sold for up to $850 or more in the United States and Japan, where reimbursement by third party payors for medical procedures is relatively high. Alternatively, the device could consist of two detachable components, of which the more expensive, thermal energy delivery component is not in contact with tissue and can be sterilized and reused, and only the less expensive cooling component, which contacts tissue or body fluids, must be discarded after a single use. Re-using the thermal energy delivery component several times can reduce the cost of the combined components to about $300 per use, making such devices affordable in countries where reimbursement for medical procedures is less than in the United States and Japan.

### BRIEF SUMMARY OF THE INVENTION

The present invention is embodied in a medical device and related method for creating a transforming effect upon tissue underlying an endothelial surface. The device and method provide for radiant energy treatment of tissue underlying an endothelial lining, avoiding damage to untargeted tissue areas, including the lining layer. The two, non-detachable component apparatus is sterile and easy to use and discard. The modular, two, detachable component apparatus allows for the sterility required for an invasive treatment at reduced cost, because the costly energy delivery component can be removed and reused, and a coacting, comparably less expensive cooling component, which contacts bodily fluids, blood and tissue, can be discarded after one use.

The apparatus aspect of this invention contemplates a thermal energy component and a separate cooling component having a cannula for receiving the thermal energy component that terminates in an energy-transmissive balloon for surrounding the thermal energy emitter. The energy-transmissive balloon provides a tissue-contacting coolant chamber. The cannula defines a coolant passageway in communication with the balloon-defined chamber. The balloon is secured to the distal end of the cannula. If laser energy is the desired thermal energy source, it is transmitted through an optical fiber or wave guide. The optical fiber or wave guide has a proximal end portion adapted to be coupled to a laser source. The cannula portion of the coolant retainer is moveably sealed around a protective sheath disposed over the optical fiber.

The laser energy device also preferably includes a handpiece secured to and located toward the proximal end of the optical fiber to facilitate handling and placement of the energy conduit in the internal duct, blood vessel, hollow organ or body cavity.

The invention relates generally to devices for applying laser and other forms of electromagnetic energy, such as incoherent white light, incoherent light of a desirable wavelength, microwave or focused ultrasound energy, through an energy transmissive, expandable balloon to transform (by, for example, mechanically cross linking collagen, denaturing, coagulating or scarring) tissue underlying endothelial linings of ducts, hollow organs or body cavities in contact with the balloon. Concomitant damage to the endothelial lining is substantially reduced by pre-cooling and/or simultaneous tissue cooling by supplying coolant to the balloon during thermal or radiant energy delivery.

A method aspect of this invention contemplates making a transforming effect upon tissue underlying an endothelial surface by providing a laser energy delivery component, including an elongate optical fiber terminating in a lateral laser emitter, providing a coolant component having a cannula for receiving the laser energy delivering component and terminating in an energy-transmissive balloon, positioning the energy-transmissive balloon adjacent tissue to be treated, supplying coolant to expand the balloon and contact the tissue, cooling the tissue for a predetermined time period, and supplying laser energy from a laser energy source through the optical fiber to the tissue through the coolant balloon for a period of time and at a laser energy intensity sufficient to transform the tissue.

While in use for radiating tissue, the energy-transmissive balloon at least partially surrounds the emitter and provides a tissue-contacting coolant chamber. The cannula defines a coolant passageway in communication with the balloon, which is secured to the distal end of the cannula. The optical fiber or wave guide has a proximal end portion with a connection to a laser source.

An apparatus to enable thermal energy to shrink tissues underlying an internal duct, blood vessel, hollow organ or body cavity is comprised of two components, one of which is movably disposed within the other component. The outer component comprises a tube or cannula to whose distal end a balloon is attached. The thermal energy delivery component is movably disposed within the outer cooling component, which can be appropriately positioned to treat the tissue at desired points through the balloon. In one embodiment of this invention, the inner component cannot be fully detached from the outer component. In another embodiment of this invention, the inner component is fully detachable from the outer component.

The thermal energy delivery component can be an optical fiber equipped to emit laser energy or high intensity incoherent light laterally or a device equipped to emit focused ultrasound or microwave energy. The balloon is filled with a cold fluid, which cools the endothelial surface of the duct, blood vessel, hollow organ or body cavity and protects it against damage while the energy passes through and produces its desired tissue effect. Such a two component apparatus would be a single use, disposable, non-detachable device, which may sell for $850, which could be afforded in the United States and Japan.

However, to reduce the cost per case of such an apparatus, and to make it affordable in less developed countries, it can be comprised of the same two components, one of which is a relatively inexpensive, outer, tissue cooling component, which is discarded after a single use, because it contacts body fluids and tissue and cannot be safely sterilized and re-used, and the other, inner, thermal energy delivery component can be a more expensive, laser energy-emitting, detachable component, which can be sterilized and reused, as it does not contact bodily tissue or fluid.

The disposable component comprises a hollow plastic or metal tube, called a cannula, on whose distal end a balloon is mounted. The balloon can be made of a flexible material, transmissive to the energy being used to create the desired tissue effect. The cannula has one or more ports, enabling fluid to be infused into or circulated through the balloon. The cannula preferably has a gasket (or elastomeric layer) and manual compression device at its proximal end, the function of which is to movably and sealingly fix the detachable, reusable, thermal energy delivery component in place within the cannula and the balloon, as known in the art. The balloon is filled with gas or liquid which is also transmissive to the wavelength of laser energy being used.

Since the thermal energy-emitting component does not contact body fluids or tissue, it can be removed from the cannula after use by loosening the compression device, safely sterilized and then re-used with another such disposable component. In an alternate embodiment of this invention, the inner, thermal energy-emitting component, while movably and sealingly fixed in place within the outer, tissue cooling component, it is prevented from being fully removed from the outer, tissue cooling component by a retaining ring, stop or other means. As a result, the entire apparatus will be disposed of after a single use.

While coherent light (laser energy), incoherent, high intensity white light, incoherent high intensity light of a desired wavelength, microwave, focused ultrasound and other forms of energy can be utilized, this invention can best be illustrated by the use of laser energy. Consequently, whenever laser energy is referred to herein, it shall apply to these other forms of thermal energy.

A fiber optic, laser energy emitting device can utilize a commercially available optical fiber that fires straight ahead, or a commercial optical fiber inside a metal tube bent at an angle up to 40 degrees or larger. However, in the treatment of many conditions, it would be desirable to emit laser energy laterally at an angle of 70° to 90° from the axis of the optical fiber.

To achieve this effect, the distal end of a commercially available optical fiber may be beveled at an angle of about 35° to 45°, preferably about 38° to 40°, and enclosed by a capillary tube to provide an air interface at the beveled surface of the optical fiber, which is necessary for total internal reflection of the laser energy laterally from the axis of the optical fiber.

Filling the balloon with air is not practical, since it could be released if the balloon ruptures, potentially creating a blood clot. If Holmium laser energy is used and the balloon is inflated, for example, with carbon dioxide (CO₂) gas, which is biocompatible in small amounts, the distal end of the optical fiber may be beveled as described above, and the need for the capillary tube may be avoided, as the laser energy will be totally internally reflected due to the refractive index of gas interface at the beveled surface of the optical fiber being significantly different from the refractive index of the optical fiber.

Alternatively, a reflective metal surface, consisting of platinum, gold, silver, copper or the like, inclined at an angle of about 40° to 50°, preferably about 44° to 46°, is positioned opposite the distal end of an ordinary, flat-ended, commercially available optical fiber to reflect the laser energy emitted from the distal end of the optical fiber at an angle of about 80° to 90°, laterally from the axis of the optical fiber.

In a preferred embodiment, the balloon is expanded with a cold gas or liquid, which cools the sensitive endothelial lining of, for example, a duct, hollow organ or body cavity prior to and during the emission of laser energy. This cools the endothelial lining of the duct, blood vessel, hollow organ or body cavity and prevents it from being thermally damaged by the laser energy, while allowing the laser energy to penetrate the tissue underlying the endothelial lining to produce its desired effect. The tissue effects of laser energy include shrinkage by photomechanical cross linkage of collagen, protein denaturization, coagulation, scarring, desiccation or vaporization.

The disposable, balloon tipped cooling component of the apparatus, which can be made of relatively inexpensive materials, may sell for about $200 and can be discarded after its use, to avoid cross-contamination and infection. The more expensive, reusable, fiber-optic component, which does not contact body fluids or tissue, may sell for about $600 and could be used, for example, ten or more times, for a cost of $60 or less per procedure. Thus, the total cost of the apparatus would be $260 or less per use, which would be affordable in countries outside the United States and Japan.

### BRIEF DESCRIPTION OF THE DRAWINGS

In the accompanying drawings that form part of the specification,

FIG. 1 is a cross-sectional view of an embodiment of a balloon-tipped cooling component of the apparatus according to an embodiment of the invention;

FIG. 2 is an external view of a side-firing laser energy delivery component of the apparatus according to an embodiment of the invention;

FIG. 3 is an enlarged, partial cross-sectional view of the laser energy delivery component shown in FIG. 2;

FIG. 4 is an enlarged, partial cross-sectional view of a laser energy delivery component according to an alternate embodiment of the invention;

FIG. 5 is an enlarged, partial cross-sectional view of the laser energy delivery component shown in FIG. 3 removably deployed within the cooling component shown in FIG. 1;

FIG. 6 is a transverse cross-sectional view of the laser energy delivery component disposed within the cooling component according to an embodiment of the present invention;

FIG. 7 is an enlarged, partial cross-sectional view of a the laser energy emitting portion of the laser energy delivery component removably deployed within the cooling component according to an alternate embodiment of the invention;

FIG. 8 is a partial, schematic view of the distal end portion of the medical device of the present invention positioned in a female urethra;

FIG. 9 is a partial, schematic view of the distal end portion of the medical device according to the present invention positioned within the annulus of the mitral valve of the heart;

FIG. 10 is a partial, schematic view of the distal end portion of the medical device according to the present invention positioned within the left ventricle of the heart;

FIG. 11 is a partial, schematic view of the distal end portion of the medical device according to the present invention removably disposed within the esophagus at the level of the esophageal sphincter;

FIG. 12 is a partial, schematic view of the distal end portion of the medical device according to the present invention removably disposed in the anus;

FIG. 13 is a partial, schematic view of the distal end portion of the medical device according to the present invention removably disposed within a bronchus of the lung;

FIG. 14 is a partial, schematic view of the distal end portion of the medical device according to the present invention removably disposed within the male urethra between the lobes of the prostate gland;

FIG. 15 is a partial, cross-sectional side view of the medical device of the present invention disposed within a movable, protective cover.

### DETAILED DESCRIPTION OF THE INVENTION

While this invention is susceptible to embodiment in many different forms, this specification and the accompanying drawings disclose only preferred forms as examples of the invention. The invention is not intended to be limited to the embodiments so described, however. The scope of the invention is identified in the appended claims.

While the references above and hereafter in this application refer to laser energy, other sources of radiant energy may be used, such as incoherent, high intensity white light or incoherent, high intensity light of a desired wavelength. Also, other forms of energy, such as microwave or focused ultrasound energy may be transmitted through balloon 12, directly ahead, at an angle or laterally from the axis of cannula 11. It is understood that wherever laser energy is mentioned herein, other sources of energy shall also apply.

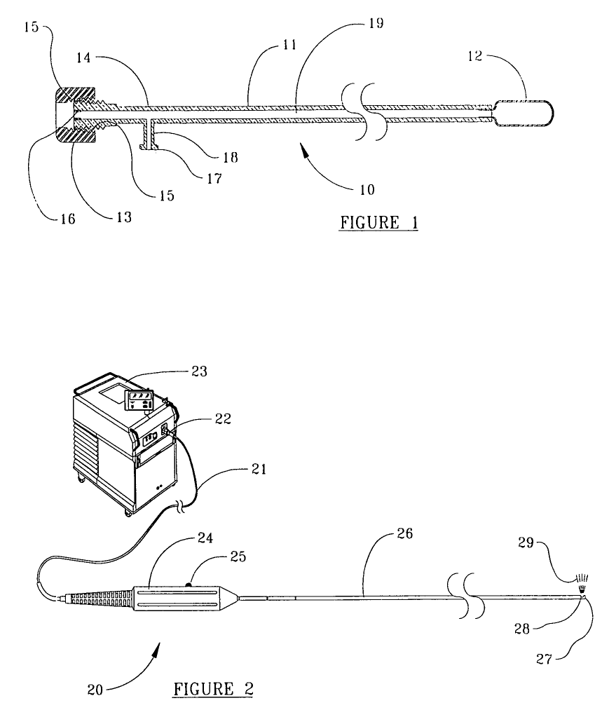

Referring to FIGS. 1 and 2, disposable includes a hollow plastic or metal tube or cannula 11 to whose distal end balloon 12 is affixed by thermal fusion, an adhesive or the like. Balloon 12 is transparent or transmissive to the wavelength of laser energy to be used to create a desired tissue effect.

Cooling component 10 has a compression coupling in the form of an externally threaded fitting 15 and a compression nut 13 for sealingly and movably grasping sheath 26 of laser energy delivery component 20 (FIG. 2). When compression cap or nut 13 is turned clockwise, a beveled inner surface 14 of nut 13 compresses threaded flanges 15 around elastomeric layer or gasket 16 to create a fluid-tight seal between cannula 11 and sheath 26 of laser energy delivery component 20.

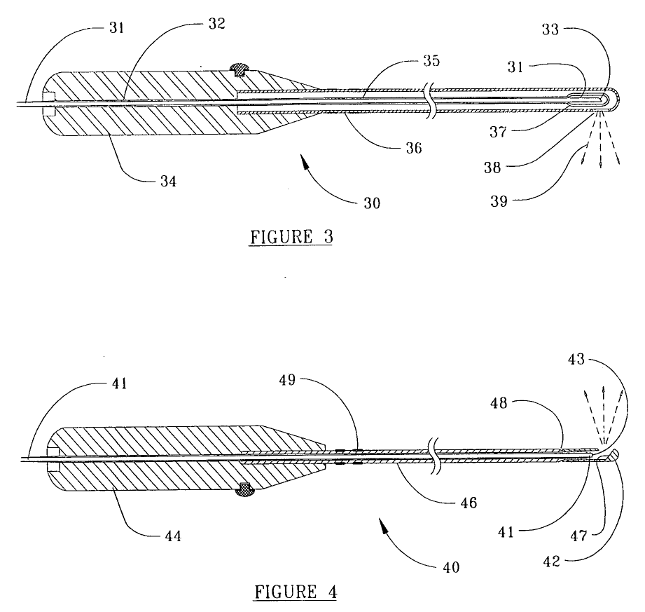

Other compression means, as known in the art, may be used to movably or removably fix sheath 26 of laser energy delivery component 20 within cannula 11 of cooling component 10. For example, one type of compression device is illustrated in FIGS. 3 and 4 of U.S. Published Application No. 2005-0113814 by Loeb, the disclosure of which is expressly incorporated herein by reference to the extent not inconsistent with the present teachings.

Cannula 11 preferably has one port for infusion of a fluid into cannula 11 to expand (or inflate) balloon 12. Alternatively, cannula 11 can have two ports (not separately shown), one of which allows fluid to be infused into and expand balloon 12, while the other port allows the fluid to exit cannula 11. In the embodiment shown in FIG. 1, a single infusion port includes a male, luer-type fitting 17 attached to tube 18, which is in fluid communication with the hollow interior of cannula 11 and balloon 12.

Preferably, the fluid infused into cannula 11 to inflate and press balloon 12 against the tissue to be treated is a gas or liquid transmissive to the wavelength of laser energy being used. In many applications, the fluid will be sterile water or saline, or a gas such as carbon dioxide (CO₂) or nitrogen. In a most preferred embodiment, the fluid is cooled and in turn cools the tissue which balloon 12 contacts, allowing the laser energy to penetrate the cooled endothelial lining of the tissue in contact with balloon 12, reducing or substantially preventing thermal damage to the sensitive endothelial lining, while the laser energy passes through and produces its desired effect on the tissue underlying the endothelial lining. If the endothelial lining of a duct, blood vessel, hollow organ or body cavity is damaged, it can cause post-operative pain and increase the risk of infection.

Laser energy penetrates the cooled endothelial lining without raising its temperature to more than about 50° C., and penetrates the underlying tissue and raises its temperature to about 500 to 60° C. to achieve its desired shrinkage or denaturing effect. For example, the area to be treated may be the urethra below the female bladder, the anus, the prostate gland below the male bladder, the esophagus in the area of the sphincter, the annulus or chordae tendonae of the mitral valve or the aorta, all of which are illustrative of shrinkage applications for the apparatus of the present invention.

Coagulation applications, where the underlying tissue is heated to about 65° C. or more while the endothelial lining is kept to a temperature of less than about 50° C., include coagulation of a lung tumor affixed to the exterior of a bronchus of the lung or the prostate to treat BPH. To create a scarring effect can require higher temperatures.

Laser energy sources which may be used with the apparatus of the present invention include, for example, diode lasers emitting at 650 to 980 nm, Nd:YAG lasers emitting at 1,064 nm, argon or KTP lasers emitting at about 438 nm or Holmium lasers emitting at about 2,100 nm. Sterile water or saline (coolant) can be used with all of the lasers cited above, except Holmium lasers, whose energy is highly absorbed by aqueous fluids. More generally, if the laser energy or source used is diode, KTP or Nd:YAG, balloon 12 is filled with a chilled liquid coolant such as saline. If the laser energy or source used is a Holmium laser is used, balloon 12 is filled with a cryogenic gas such as expanded CO₂ or expanded nitrogen.

Balloon 12 may be made of a substantially compliant or substantially non-compliant, flexible material which is of a desirable thickness, tensile strength and substantially transmissive to the radiant energy being emitted, including materials such as natural rubber, a polyurethane, a polyethylene, a polyethylene terephthalate, a polyester, a co-polyester, a polyvinyl chloride, a copolymer of vinyl chloride and vinylidene chloride and composites thereof.

FIG. 2 illustrates laser energy delivery component 20 of the apparatus of the present invention, the distal end of which is constructed to emit light energy laterally from the axis of optical fiber 21. Optical fiber 21 extends from connector 22, which is in optical communication with laser 23. Optical fiber 21 extends through and is fixed within handpiece 24 by an adhesive or the like. Button 25 on handpiece 24 indicates the direction in which the laser energy will be emitted. In the embodiment shown, laser energy will be emitted from the same side of cannula 26 indicated by button 25. In an alternate embodiment, laser energy can be emitted in the opposite direction from the orientation of button 25 on handpiece 24. When the operator puts his/her index finger on button 25, the index finger will be pointing in the direction of the laser energy emission.

optical fiber 21 also extends through protective plastic or metal sheath 26, the purpose of which is to protect optical fiber 21 while it is advanced into place in the body. Sheath 26 is fixedly attached to handpiece 24 by an adhesive or the like and extends distally from handpiece 24. In the embodiment shown, distal end 27 of sheath 26 has a blunt ended shape to prevent damage to the duct, blood vessel, hollow organ or body cavity. Alternatively, distal end 27 of sheath 26 may be pointed, may be a sharp, syringe needle-like shape or be of any other shape. Offset from and proximal to distal end 27 of cannula 26 is an opening or port 28 in sheath 26 for emission of laser energy in the direction shown by lines 29, by a means described in detail with reference to FIG. 3. Sheath 26 may be made of medical grade stainless steel or any flexible or semi-rigid biocompatible plastic.

FIG. 3 illustrates a preferred embodiment of the thermal energy delivery component 30 of the apparatus of the present invention. In this embodiment, optical fiber 31 extends through handpiece 34 and is fixed therein by adhesive 32. The proximal end of metal or plastic sheath 36 is also fixed within the distal end of handpiece 34 by adhesive 32. Optical fiber 31 also extends through sheath 36, and the distal end surface 33 of optical fiber 31, the distal end portion buffer coating 35 having earlier been removed, has been beveled at an angle of about 35° to 45°, preferably about 38° to 40°. Distal end surface 33 is encased in a closed ended capillary tube 37, the proximal end of which is sealed about bare optical fiber 31 by thermal fusion or an adhesive, as known in the art, and whose distal end has been closed by thermal fusing. Capillary tube 37 provides an air interface opposite the beveled distal end surface 33 of optical fiber 31, which is necessary for substantially total internal reflection of light energy laterally from the axis of optical fiber 31.

Preferably, sheath 36 is coextensive with optical fiber 31 (i.e., extends from the distal end of handpiece 34 fully over optical fiber 31), to provide superior stability and a smooth, contiguous outer surface. As can be seen, opening or port 38 in sheath 36 allows laser energy to be emitted laterally, as indicated by arrows 39.

FIG. 4 illustrates an alternate embodiment of the laser energy delivery component 40 of the apparatus of the present invention. As shown, the distal end of optical fiber 41 extends partially through tip 42 and presents a substantially flat end shape 43, such that laser energy will be emitted directly ahead (i.e., in an axial direction). Tip 42 has been attached to recess 48 in the distal end portion of a plastic or metal sheath 46 by an adhesive, crimping or both (not separately shown). While tip 42 may be attached directly to optical fiber 41, attaching tip 42 to a recess 48 in sheath 46 provides a substantially smooth outer surface of energy emitting component 40. However, tip 42 can be attached to optical fiber 41 by any other means known in the art.

Tip 42 has a beveled surface 47 opposite the flat distal end face 43 of optical fiber 41, as described in co-owned U.S. Pat. No. 5,649,924 to Everett et al, which is expressly incorporated herein by reference. Beveled surface 47 is inclined at an angle of about 40° to 50°, preferably about 44° to 46°, to reflect the laser energy as shown at an angle of about 80° to 90°. Tip 42 is may be made of stainless steel, whose exterior has been plated with gold, silver or copper with a thickness of at least 5 thousandths of an inch. Silver provides almost the same reflectivity of gold but is less expensive, and silver has higher reflectivity than copper.

Preferably, beveled surface 47 of tip 42 can have a recess into which an insert of copper, silver or gold (not separately shown), with a thickness of at least 10 thousandths of an inch may be fixed, by an adhesive, force fit or other means known in the art. In a most preferred embodiment, tip 42 is made entirely of copper, silver or gold for enhanced resistance to degradation by prolonged exposure to laser energy. Since silver is more reflective than copper and is less expensive than gold, silver is preferred.

Alternatively, tip 42 can be made of a heat resistant plastic, the exterior of which is similarly plated with gold, silver or copper. Alternatively, plastic tip 42 can have a recess 48, into which a gold, silver or copper insert, as described above, may be fixed in place by an adhesive, force fitting or both, forming beveled surface 47, which is inclined at an angle of about 40° to 50° C., preferably of about 44° to 46° C.

Markings 49 on the exterior of sheath 46 or, if sheath 46 is eliminated, on optical fiber 41, indicate to the user the position of the distal end of tip 42 within balloon 12 of coolant retainer component 10 (FIG. 1).

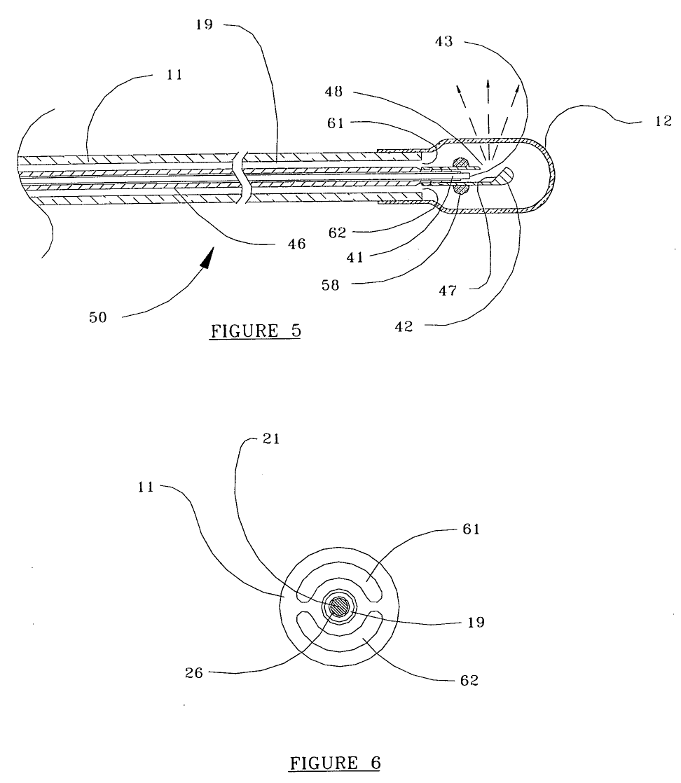

FIGS. 5 and 6 illustrate the distal end portion of the two component medical device 50 according to the present invention. The distal end portion of laser energy delivery component 40 is disposed within the distal end portion of the balloon-tipped cannula component 10. However, in this embodiment, plastic cannula 11 has been extruded with a central channel for optical fiber 41 and-sheath 46, and two kidney-shaped channels 61 and 62 for infusing fluid in and out of cannula 11 and balloon 12, as shown in FIG. 6. Fluid is infused in and out of cannula 11 and balloon 12 by input and output ports (not separately shown) in cannula 11, which are in fluid communication with channels 61 and 62, respectively. Alternatively, channels 61 and 62 can be round, crescent or of any other cross-sectional shape. Means for circulating fluid through cannula 11 and balloon 12 at a constant rate, pressure and/or temperature are known in the art and are not described herein.

In one embodiment, energy delivery component 20 and coolant component 10 have complementary dimensions to prevent detachment. Delivery component 20 preferably includes a protrusion small enough to allow insertion into coolant component 10 but large enough to prevent complete withdrawal. For example, ring 58, which is fixedly attached by an adhesive or the tube on the exterior surface of sheath 46 prevents laser energy delivery component 40 of FIG. 4 from being removed from cannula 11, of cooling component 10 of FIG. 1, as ring 58 has a larger outside diameter than channel 19 of cooling component 10 (FIG. 1), making laser energy delivery component 40 (FIG. 4) non-detachable from cannula/balloon cooling component 10 of FIG. 1. Other means may be used instead of ring 58 to prevent the removal of laser energy delivery component 40 from cannula 11. If it is desired that fiber-optic component 40 of FIG. 4 be completely detachable from cannula component 11 of FIG. 1, as provided in the two component, detachable embodiment of the present invention, ring 58 or other retaining are not provided.

FIG. 6 illustrates an alternative embodiment of cannula 11 of FIG. 1. In this embodiment, cannula 11 is extruded with central channel 19 through which optical fiber 21 movably extends. Fluid is infused through channel 61, circulates through balloon 12 (not separately shown) and exits through channel 62.

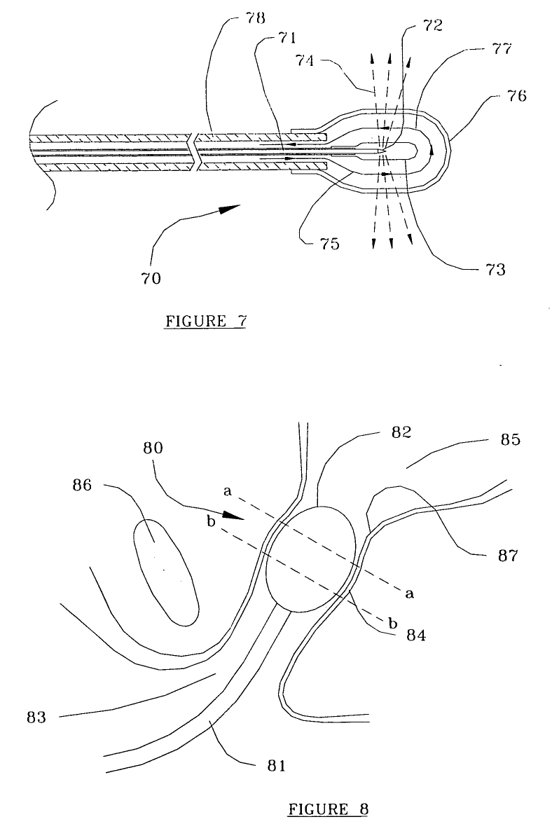

FIG. 7 illustrates an alternate embodiment 70 of a laser energy delivery component 30 of FIG. 3 according to the present invention. In this embodiment, there is no sheath over optical fiber 71. Instead of the distal end of optical fiber 31 being beveled into a single, prism-like surface 33 as shown in FIG. 3, the distal end of optical fiber 71 is instead beveled into a conical shape 72, as described in co-owned U.S. Pat. No. 5,242,438 to Saadatmanesh, which is expressly incorporated herein by reference. The distal end portion of optical fiber 71 is encased by distal close-ended capillary tube 73 to create an air interface opposite the surface of conical shape 72. Laser energy is emitted in a 360-degree arc, laterally from the axis of optical fiber 71, as indicated by arrows 74. Arrow 75 indicates the path of coolant fluid into balloon 76, and arrow 77 indicates the path of coolant fluid circulating through balloon 76 and out of cannula 78. If Holmium laser energy is to be used, balloon 76 is filled with CO₂ gas, and capillary tube 73 may be eliminated. However, to prevent the sharp point 72 of optical fiber 71 from inadvertently puncturing balloon 76, capillary tube 73 is best retained.

In this embodiment, the fluid optionally comprises saline in which light reflecting particles are suspended, as described in U.S. Pat. No. 4,612,938 to Dietrich, which is hereby expressly incorporated by reference. Preferred light reflecting particles are microscopic, inert quartz particles, known as fumed silica, such as Cab-O-Sil made by Cabot Corporation of Boston, Mass., a suspension of albumen microspheres with a diameter of less than 10 microns at a concentration of less than 25%, or a commercially available high fat intravenous fluid suspension. The light reflecting particles reflect the laser energy, creating a more uniform emission pattern. Such light reflecting particles can be incorporated into the liquid used to inflate the balloon of any of the embodiments of the invention, and the liquid may be cooled to cool and protect the endothelial surface of the duct, hollow organ or body cavity.

In FIG. 8, the distal end portion 80 of the apparatus of the present invention is disposed within female urethra 83 at the level of sphincter 84 below bladder 85, with a cold fluid infused through cannula 81 having expanded balloon 82. Pubic bone 86 is shown to the left. Dotted lines a-a and b-b indicate the desired positions for emission of laser energy. In this embodiment, two markings 49, as shown in FIG. 4, are made on sheath 26, 36 or 46 of reusable laser energy delivery components 20, 30 or 40 shown in FIG. 2, 3 or 4, respectively. When the markings reach the proximal end of compression nut 13 at the proximal end of cannula 11 (FIG. 1), these marking each indicate that the laser emission port 28, 38 or 48 of reusable fiber-optic component 20, or 40 (FIG. 2, 3 or 4, respectively) has reached dotted line positions a-a or b-b within balloon 12 of FIG. 1. Any number of positions for the emission of laser energy may be employed, depending on the size of the area to be treated, with the number and position of markings on sheath 26, 36 and 46 of FIGS. 20, 30 and 40 corresponding thereto.

Balloon 82 is inflated with a cold fluid during or, preferably, for at least about five seconds prior to and during the emission of laser energy, to cool the endothelial lining 87 of urethra 83 in contact with balloon 82. Laser energy may be emitted at about 2 to 25 watts, preferably at about 5 to 15 watts, for about 5 to 60 seconds, preferably for about 10 to 30 seconds, by rotating handpiece 24 and using button 25 on the handpiece 24 of the device of FIG. 2 to direct the laser energy to, for example, each of 12, 3, 6 and 9 o'clock at position a-a to shrink urethral sphincter 84, repeating the above procedure at each of 12, 3, 6 and 9 o'clock at position b-b, to treat female stress urinary incontinence.

If the laser energy emitter is the 360° lateral laser energy emitting device 70 shown in FIG. 7, button 25 is eliminated, and laser energy is emitted in a 360° arc at about 2 to 15 watts, preferably about 5 to 15 watts, without rotating handpiece 24, for about 20 to 240 seconds, preferably for about 40 to 120 seconds, at each of positions a-a and b-b, using markings 49 of FIG. 4, as described above. Only one lasing position or more than two may also be used.

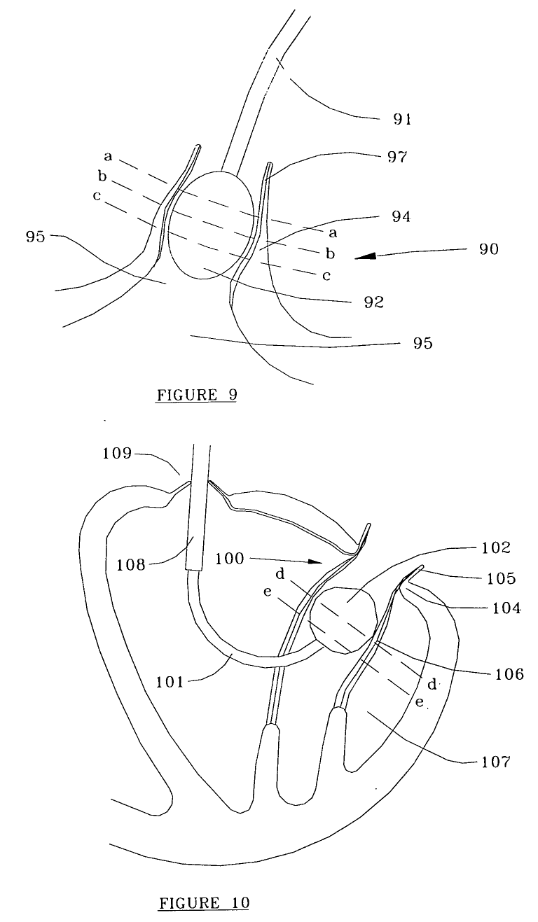

FIG. 9 illustrates the distal end portion of apparatus 90 disposed within the annulus 93 and leaflets 94 of mitral valve 95, with a cold fluid infused through cannula 91 having inflated balloon 92. Laser energy is emitted at positions a-a, b-b and c-c at the power levels and for the time periods described above in FIG. 8, with the cold fluid cooling endothelial lining 97 of mitral valve 95 and leaflets 94 during and, preferably, for at least about 5 seconds before and during the emission of laser energy to shrink annulus 93 and, if desired, leaflets 94 to improve the closure of mitral valve 95. Any other selection of lasing positions may be also used.

FIG. 10 illustrates the distal end portion of apparatus 100 disposed within the left ventricle 107 of the heart. If prolapse of mitral valve 104 is caused or contributed to by stretching of chordae tendonae 106, the distal end portion of apparatus 100 may be positioned in left ventricle 107 of the heart by inserting guiding catheter 108 through aortic valve 109, as known in the art. Cannula 101 may have been thermally preformed into the curved shape shown, may be articulated by wires, as known in the art, or may be positioned by any other means. Fluid is infused into balloon 102, pressing balloon 102 against chordae tendonae 106. Laser energy is emitted as described above at positions d-d and e-e to shrink and tighten chordae tendonae 106, which in turn cause leaflets 105 of mitral valve 103 to close tighter.

Preferably, for at least about 5 seconds before and during the emission of laser energy, a cold fluid is infused into cannula 101 and balloon 102, or circulated through cannula 101 and balloon 102, to cool chordae tendonae 106 while the shrinkage is created. After deflating balloon 102, cannula 101 and balloon 102 may be withdrawn into catheter 109, and catheter 109 may then be withdrawn from the body.

If the emission of laser energy in pulses, with a duration of about 0.2 to 0.4 seconds, is synchronized with the patient's ECG to occur during systole, when chordae tendonae 106 are relaxed, up to 30% shrinkage of charade tendonae 106 has been shown to occur, whereas the same amount of laser energy emission occurs during diastole, when chordae tendonae 106 are tightly stretched to close leaflets 105.

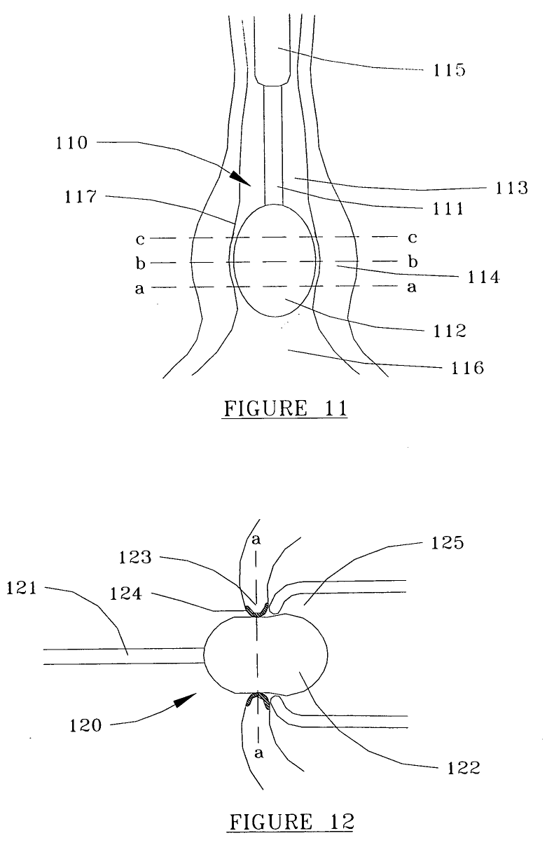

In FIG. 11, the distal end portion apparatus 110 is shown positioned in the esophagus 113 in the area of the esophageal sphincter 114. Apparatus 110 is inserted, with balloon 112 deflated, through a delivery catheter or a channel of endoscope 115. Cold fluid is infused through cannula 111 to inflate balloon 112, or circulated through balloon 112, during or, preferably, for at least about 5 seconds before and during the emission of laser energy at positions a-a, b-b and c-c, or a greater or lesser number of positions, at the energy levels and for the time periods described above, to shrink the esophageal sphincter to treat gastroesophageal reflux disease or GERD. Cold fluid infused through cannula 111 inflates balloon 112 cools and prevents thermal damage to endothelial lining 117 of sphincter 114.

FIG. 12 illustrates the distal end portion of apparatus 120 with cannula 121 and balloon 122 positioned in anus 123. Cold fluid infused into or circulated through balloon 122 during or, preferably, for at least about 5 seconds before and during the emission of laser energy at position a-a, at the energy levels and for the time period described above, cools and prevents thermal damage to endothelial lining 124 of anus 123, while the laser energy shrinks the tissue of anus 123 to treat fecal incontinence. More than one lasing position may be used, if desired.

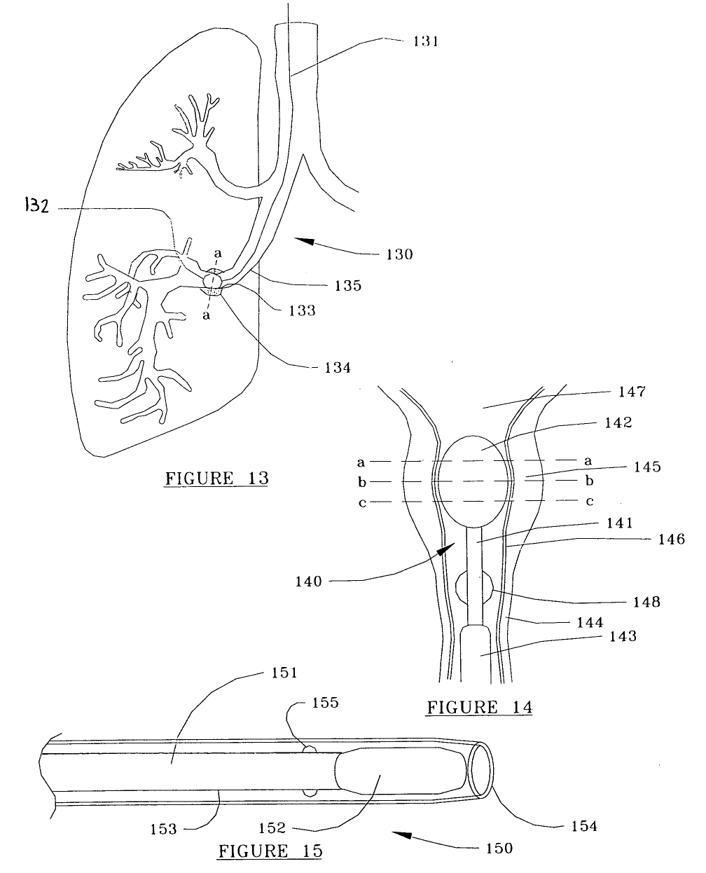

FIG. 13 shows the distal end portion of apparatus 130 with cannula 131 and balloon 132 positioned in the bronchus 133 of the lung 135 of a person with a tumor 134 surrounding bronchus 133. A cold fluid is infused into or circulated through cannula 131 to inflate balloon 132 and cool endothelial lining 135 of bronchus 133, preferably, for at least about 5 seconds before and during the emission of laser energy at the powers and for the time periods described above at position a-a or additional positions, depending on the size of tumor 134. The laser energy lethally coagulates or denatures the proteins of tumor 134 to treat lung cancer.

FIG. 14 illustrates the distal end portion of apparatus 140 with cannula 141 and balloon 142 extending distally from a catheter or a channel of endoscope 143 and positioned in the male urethra 144 within prostate gland 145. A cold fluid may be infused into cannula 141 to inflate balloon 142 and cool the endothelial lining 146 of urethra 144, while laser energy passes through the shrink, denature proteins or coagulate prostate 145 to treat benign prostatic hyperplasic or BPH. Balloon 142 is advanced to its position proximal to bladder neck 147, having passed over veru montaneum 148. Lines a-a, b-b and c-c illustrate some of the positions at which laser energy may be emitted at the energy levels and for the time periods described above to shrink or denature the proteins of the prostate or, at higher energy levels for longer time periods, to coagulate the prostate to treat BPH.

FIG. 15 illustrates the distal end portion of apparatus 150, with cannula 151 and balloon 152 movably disposed within retractable cover 153. Optionally, as shown in FIG. 15, distal end 154 of retractable cover 153 may be slightly curved inwardly to form a less traumatic distal end. Alternatively, the distal end of cannula 151 may not be curved inwardly (not separately shown). Cover 153 prevents damage to balloon 152 when apparatus 150 is inserted through a channel of an endoscope (not shown) or directly into a duct, blood vessel, hollow organ or body cavity. When cover 153 is retracted, narrowed distal end cannot pass beyond ring 155, at which point the operator knows, balloon 152 is fully exposed and can be inflated with a cold fluid and laser energy emitted as described above. Flanges and other means known in the art can be employed to prevent cover 153 from being retracted further than necessary to fully expose balloon 152.

Alternatively, markings (not separately shown) outside the body on cannula 151 can indicate to the operator when cover 153 has been extended fully over balloon 152 and when cover 153 has been retracted and balloon 142 is fully exposed.

### EXAMPLE

A group of medical devices were constructed according to embodiments shown in FIGS. 1 and 3. Specifications for selected features are presented below in Table I.

TABLE I

| | |
|---|---|
| Device | Specification |

Laser Energy Emitting Component:

| | |
|---|---|
| fiber optic | 365 to 550 micron case diameter |
| | 3 meters in length |
| sheath material | medical grade stainless steel or |
| | PEEK |
| sheath outside diameter | 1.5 to 2.33 mm |
| emitter configuration | Beveled, prism-like optical fiber |
| capillary tube | Fused silica |
| preferred laser type | Diode |

Coolant Retainer/Balloon:

| | |
|---|---|
| cannula length | 10 to 75 cm |
| (handpiece to balloon) | |
| cannula outside diameter | 2 to 3 mm |
| balloon material | silicone |
| balloon diameter (inflated) | 5 to 80 mm |
| port(s) | Luer configuration |

The meetings and sizes of the example devices vary by the particular medical application. The example devices provide a reusable higher-cost thermal energy delivery component and a relatively lower cost, disposable coolant component. These devices can be made non-detachable as a single use, disposable device, or detachable to enable the more costly laser energy delivery component to be reused and the lower cost, outer, cooling component to be discarded after one use. Such devices enable the treatment of tissues underlying internal ducts, blood vessels, hollow organs and body cavities. with protective cooling for their endothelial linings.

Numerous variations and modifications of the embodiments described above can be effected without departing from the spirit and scope of the novel features of the invention. It is to be understood that no limitation with respect to the specific apparatus illustrated herein is intended or should be inferred. It is, of course, intended to cover by the appended claims, all such modifications as fall within the scope of the claims.

What is claimed is:

1. A modular device suitable for creating a transforming effect upon tissue underlying an endothelial surface, the device comprising:
a laser energy delivery component including an optical fiber terminating in a lateral laser emitter, said optical fiber having a proximal end portion adapted to be coupled to a laser source;
a disposable coolant component having a cannula for receiving said laser energy component and terminating in an energy-transmissive balloon for surrounding said emitter and providing a tissue-contacting coolant chamber, said cannula defining a coolant passageway in communication with said balloon and said balloon being fixed to a distal end of said cannula,
wherein said cannula is moveably sealed around said optical fiber.

2. The device according to claim 2 wherein said lateral laser emitter comprises a distal end of said optical fiber beveled at an angle in the range of about 35 to 45 degrees and enclosed in a capillary tube to create an environment opposite the beveled surface of a sufficiently different refractive index than that of the optical fiber to cause the radiant energy to, be internally reflected laterally from the axis of said optical fiber.

3. The device according to claim 1 wherein said optical fiber terminates in a end portion with a distal end beveled at an angle in the range of about of about 35 to 45 degrees and sealed within a capillary tube, such that laser energy is emitted through said balloon at an angle of about 70 to about 90 degrees from the axis of a distal end portion of said fiber when said optical fiber is coupled to a laser energy source and said laser delivery component is movably disposed within the coolant component.

4. The device according to claim 1 wherein said lateral emitter comprises a reflector positioned generally axially aligned with said optical fiber.

5. The device according to claim 4 wherein said reflector includes a reflective coating.

6. The device according to claim 1 wherein said lateral laser emitter comprises:
a tip defining a cavity within which a distal end of said optical fiber is received, the cavity having a distal end wall inclined of an angle of about 40° to 50° and wherein said end wall is reflective to the wavelength of laser energy being used for reflecting a laser energy beam emitted coaxially with said distal end of said optical fiber;
a laterally open aperture to said cavity, said aperture being open to fluid communication from outside said tip through said aperture into said cavity.

7. The device according to claim 6 wherein said distal end wall comprises a reflective insert.

8. The device according to claim 6 wherein said tip is constructed of a reflective material.

9. The device according to claim 6 wherein the end wall is inclined of an angle in the range of about 44° to 46°.

10. The device according to claim 1 wherein said coolant passageway is in communication with a source of coolant.

11. The device according to claim 1 wherein said coolant passageway is operably connected to a source of coolant through an access opening formed in the material of said cannula.

12. The device according to claim 1 wherein said cannula is moveably sealingly held on said proximal end portion by a seal lock between said cannula and said optical fiber.

13. The device according to claim 1 wherein said balloon is constructed of a substantially compliant polymeric material.

14. The device according to claim 1 wherein said balloon is constructed of a substantially non-compliant material.

15. The device according to claim 1 wherein said balloon is constructed of a material selected from the group consisting of a silicone, latex, natural rubber, a polyurethane, a polyethylene, a polyethylene terephthalate, a polyester, a copolyester, a polyvinyl chloride, a copolymer of vinyl chloride, vinylidene chloride and composites thereof.

17. The device according to claim 1 including a source of fluid in fluid communication with said coolant passageway for filling said balloon and cooling said chamber and wherein said fluid is selected from the group consisting of expanded carbon dioxide gas, expanded nitrogen gas, chilled water and chilled saline.

18. The device according to claim 1 wherein a distal portion of said energy delivery component is movably and sealingly disposed within said coolant retainer but not detachable therefrom.

19. The device according to claim 1 wherein said laser energy delivery component and said coolant component have complementary dimensions to prevent detachment of said delivery component from said coolant component.

20. The device according to claim 1 wherein said laser energy delivery component has a retaining protrusion dimensioned to prevent detachment from said coolant component.

21. The device according to claim 1 wherein said retaining protrusion is a retaining ring fixed to a distal end portion of said energy delivery component.

22. The device according to claim 1 wherein a distal portion of said energy delivery component is movably and sealingly disposed within said coolant retainer and detachable therefrom.

23. The device according to claim 1 adapted to deliver a thermal energy selected from the group consisting of laser energy, substantially incoherent light, incoherent light of a predetermined wavelength range and microwave energy.

24. The device according to claim 1 wherein said laser energy delivery component includes a tactile indicator for an aim of the emitter.

25. A modular device suitable for creating a transforming effect upon tissue underlying an endothelial surface, the device comprising:
a thermal energy delivery component including an optical fiber terminating in a lateral emitter, said optical fiber having a proximal end portion adapted to be coupled to a thermal energy source;
a disposable coolant component having a cannula for receiving said energy component and terminating in an energy-transmissive balloon for surrounding said emitter and providing a tissue-contacting coolant chamber, said cannula defining a coolant passageway in communication with said balloon and said balloon being fixed to a distal end of said cannula,
wherein said cannula is moveably sealed around said optical fiber and wherein said energy delivery component and said coolant component have complementary dimensions to prevent detachment of said delivery component from said coolant component.

26. A method for making a transforming effect upon tissue underlying an endothelial surface, comprising the steps of:
(a) providing a thermal energy delivery component including an optical fiber terminating in a lateral thermal energy emitter, said optical fiber having a proximal end portion coupled to a source of thermal energy;
(b) providing a coolant component having a cannula for receiving said laser delivery component and terminating in an energy-transmissive balloon for surrounding said emitter and creating a tissue-contacting coolant chamber, said cannula defining a coolant passageway in communication with said balloon and said balloon being fixed to a distal end of said cannula;
(c) positioning said energy-transmissive balloon adjacent tissue to be treated, said emitter being at least partially surrounded by said balloon;
(d) supplying coolant through said passageway to expand said balloon and contact said tissue;
(e) cooling said tissue for a predetermined time period; and
(f) supplying thermal energy from said thermal energy source through said optical fiber to said tissue through said coolant balloon for a period of time and at a thermal energy intensity sufficient to transform said tissue.

27. The method according to claim 26 wherein said tissue to be treated is selected from the group consisting of tissue underlying the endothelial surface of a duct, blood vessel, hollow organ and body cavity.

28. The method according to claim 27 wherein said transforming effect is to reduce the volume of tissue underlying said duct, hollow organ or body cavity by a increasing the density of said underlying tissue or by creating localized scarring of said underlying tissue.

29. The method according to claim 26 wherein said transforming effect is selected from the group consisting of shrinking, denaturizing, coagulating, scarring, desiccating and vaporizing said underlying tissue.

30. The method according to claim 26 wherein said thermal energy is laser energy having a wavelength range selected from the group consisting of 400 to 600 nanometers, 600 to 1,000 nanometers, 1,000 to 1,800 nanometers and 1,800 to 2,200 nanometers, and wherein the pattern of laser emission is selected from the group consisting of continuous wave, pulses with a duration of 200 to 500 microseconds, pulses with a duration of 500 to 1,000 microseconds, pulses with a duration of 1 to 500 milliseconds, and pulses with a duration of 500 to 2,000 milliseconds, and wherein said range of repetition rate is selected from the group consisting of 1 to 30 per second, 30 to 80 per second and 80 to 200 per second.

31. The method according to claim 26 wherein said thermal energy is selected from the group consisting of laser energy, substantially incoherent light, incoherent light of predetermined wavelength range and microwave energy.

32. The method according to claim 26 wherein said energy delivery component has a retaining protrusion dimensioned to prevent detachment from said coolant component.
